(19) United States
(12) Patent Application Publication
(10) Pub. No.: US 2006/0035877 A1
(43) Pub. Date: Feb. 16, 2006

(54) Method and compositions for treating pulmonary diseases

(76) Inventors: Peter N. Goodfellow, Harlow (GB); Richard Nieman, Philadelphia, PA (US); Theodore J. Torphy, Bryn Mawr, PA (US)

Correspondence Address: GLAXOSMITHKLINE;Corporate Intellectual Property - UW2220, P.O. Box 1539, King of Prussia, PA, 19406-0939, US

(21) Appl. No.: 11/217,532

(22) Filed: Sep. 1, 2005

Related U.S. Application Data
(63) Continuation of application No. 10/847,223, filed on May 17, 2004, now abandoned.
Application No. 10/847,223 is a continuation of application No. 10/129,311, filed on May 2, 2002, now abandoned.
(60) Provisional application No. 60/163,158, filed on Nov. 2, 1999.

(51) Int. Cl. A61K 31/573; A61K 31/277

(52) U.S. Cl. 514/171; 514/521

(57) ABSTRACT

This invention relates to treating pulmonary diseases such as chronic obstructive pulmonary disease or asthma by administering a phosphodiesterase 4 inhibitor in combination with anti-inflammatory corticosteriod.

### AREA OF THE INVENTION

This invention relates compositions and methods for preventing or reducing the onset of symptoms of pulmonary diseases, or treating or reducing the severity of pulmonary diseases. In particular it relates to compositions and methods for treating pulmonary diseases mediated by phosphodiesterase 4 (PDE4) by administering a PDE4 inhibitor with an anti-inflammatory corticosteroid.

### BACKGROUND OF THE INVENTION

Identification of novel therapeutic agents for treating pulmonary diseases is made difficult by the fact that multiple mediators are responsible for the development of the disease. Thus, it seems unlikely that eliminating the effects of a single mediator could have a substantial effect on all three components of chronic asthma. An alternative to the “mediator approach” is to regulate the activity of the cells responsible for the pathophysiology of the disease.

One such way is by elevating levels of cAMP (adenosine cyclic 3′,5′-monophosphate). Cyclic AMP has been shown to be a second messenger mediating the biologic responses to a wide range of hormones, neurotransmitters and drugs; [Krebs Endocrinology Proceedings of the 4th International Congress Excerpta Medica, 17-29, 1973]. When the appropriate agonist binds to specific cell surface receptors, adenylate cyclase is activated, which converts Mg⁺²-ATP to cAMP at an accelerated rate. Cyclic AMP modulates the activity of most, if not all, of the cells that contribute to the pathophysiology of extrinsic (allergic) asthma. As such, an elevation of cAMP would produce beneficial effects including: 1) airway smooth muscle relaxation, 2) inhibition of mast cell mediator release, 3) suppression of neutrophil degranulation, 4) inhibition of basophil degranulation, and 5) inhibition of monocyte and macrophage activation. Hence, compounds that activate adenylate cyclase or inhibit phosphodiesterase should be effective in suppressing the inappropriate activation of airway smooth muscle and a wide variety of inflammatory cells. The principal cellular mechanism for the inactivation of cAMP is hydrolysis of the 3′-phosphodiester bond by one or more of a family of isozymes referred to as cyclic nucleotide phosphodiesterases (PDEs).

It has been shown that a distinct cyclic nucleotide phosphodiesterase (PDE) isozyme, PDE4, is responsible for cAMP breakdown in airway smooth muscle and inflammatory cells. [Torphy, “Phosphodiesterase Isozymes: Potential Targets for Novel Anti-asthmatic Agents” in New Drugs for Asthma, Barnes, ed. IBC Technical Services Ltd., 1989]. Research indicates that inhibition of this enzyme not only produces airway smooth muscle relaxation, but also suppresses degranulation of mast cells, basophils and neutrophils along with inhibiting the activation of monocytes and neutrophils. Moreover, the beneficial effects of PDE 4 inhibitors are markedly potentiated when adenylate cyclase activity of target cells is elevated by appropriate hormones or autocoids, as would be the case in vivo. Thus PDE 4 inhibitors would be effective in the lung, where levels of prostaglandin E₂ and prostacyclin (activators of adenylate cyclase) are elevated. Such compounds would offer a unique approach toward the pharmacotherapy of bronchial asthma and possess significant therapeutic advantages over agents currently on the market.

In addition, it could be useful to combine therapies in light of the fact that the etiology of many pulmonary diseases involves multiple mediators. In this invention there is presented the combination of a PDE 4 inhibitor and an anti-inflammatory corticosteroid, particularly one delivered by inhalation, for treating pulmonary diseases. This combination is particularly useful for treating chronic obstructive pulmonary disease (COPD) or asthma.

### SUMMARY OF THE INVENTION

In a first aspect this invention relates to a method for treating a pulmonary disease in a mammal by administering to a patient in need thereof an effective amount of a PDE 4-specific inhibitor and an effective amount of a steroidal anti-inflammatory agent wherein the drugs are administered concomitantly together or separately and sequentially where the sequential administration is close in time or remote in time.

In a second aspect this invention relates to a composition for treating a pulmonary disease in a mammal comprising an effective amount of a PDE4-specific inhibitor, an effective amount of a steroidal anti-inflammatory agent and a pharmaceutically acceptable excipient.

### DETAILED DESCRIPTION OF THE INVENTION

The combination therapy contemplated by this invention comprises administering a PDE4 inhibitor with a steroidal anti-inflammatory agent to prevent onset of a pulmonary disease event or to treat an existing condition. The compounds may be administered together in a single dosage form. Or they may be administered as two different formulations which may be the same or different. To illustrate, both drugs may be provided separately as oral formulations, or one may be an oral preparation and the other as an inhalant, or both may be provided in an inhaled dose form. They may be administered at the same time. Or they may be administered either close in time or remotely, such as where one drug is administered in the morning and the second drug is administered in the evening.

The combination may be used prophylactically or after the onset of symptoms. In some instances the combination(s) may be used to prevent the progression of a pulmonary disease or to arrest the decline of a function, such as lung function.

The PDE4-specific inhibitor useful in this invention may be any compound that is known to inhibit the PDE4 enzyme or which is discovered to act in as PDE4 inhibitor, and which are only PDE4 inhibitors, not compounds which inhibit other members of the PDE family as well as PDE4. Generally it is preferred to use a PDE4 antagonists which has an IC₅₀ ratio of about 0.1 or greater as regards the IC₅₀ for the PDE4 catalytic form which binds rolipram with a high affinity divided by the IC₅₀ for the form which binds rolipram with a low affinity.

PDE inhibitors used in treating inflammation and as bronchodilators, drugs like theophylline and pentoxyfyllin, inhibit PDE isozymes indiscriminately in all tissues. These compounds exhibit side effects, apparently because they non-selectively inhibit all 5 PDE isozyme classes in all tissues. The targeted disease state may be effectively treated by such compounds, but unwanted secondary effects may be exhibited which, if they could be avoided or minimized, would increase the overall therapeutic effect of this approach to treating certain disease states. For example, clinical studies with the selective PDE 4 inhibitor rolipram, which was being developed as an antidepressant, indicate it has psychotropic activity and produces gastrointestinal effects, e.g., pyrosis, nausea and emesis.

For purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

Initial experiments were conducted to establish and validate a [3H]-rolipram binding assay. Details of this work are given in Example 1 below.

To determine whether both the high affinity binding activity and the low affinity binding activity resided in the same gene product, yeast-were transformed by known methods and the expression of recombinant PDE 4 was followed over a 6 hour fermentation period. Western blot analysis using an antibody directed against PDE 4 indicated that the amount of PDE 4 expressed increased with time, reaching a maximum after 3 hour of growth. In addition, greater than 90% of the immunoreactive product was in the high speed (100,000×g) supernatant of yeast lysates. [³H]R-(−)-Rolipram binding and PDE activity were monitored along with protein expression. PDE 4 activity was co-expressed with rolipram binding activity, indicating that both functions exist on the same gene product. Similar to results with the Western plot analysis, greater than 85% of the rolipram-inhibitable PDE activity and [³H]-rolipram binding activity was found to be present in the yeast supernatant fraction.

Overall, most of the recombinant PDE 4 expressed in this system exists as LPDE 4 and only a small fraction as HPDE 4. Consequently, inhibition of recombinant PDE 4 catalytic activity primarily reflects the actions of compounds at LPDE 4. Inhibition of PDE 4 catalytic activity can thus be used as an index of the potency of compounds at LPDE 4. The potency of compounds at HPDE 4 can be assessed by examining their ability to compete for [³H]R-rolipram. To develop SARs for both the low affinity and high affinity rolipram binding sites, the potencies of selected compounds were determined in two assay systems. Results from experiments using standard compounds were tabulated. As expected, certain compounds were clearly more potent in competing with [³H]-rolipram at the site for which rolipram demonstrated high affinity binding as compared with the other site, the one at which rolipram is a low affinity binder. SAR correlation between high affinity binding and low affinity binding was poor and it was concluded that the SAR for inhibition of high affinity [³H]-rolipram binding was distinct from the SAR for binding to the low affinity rolipram binding site.

It is now known that there are at least two binding forms on human monocyte recombinant PDE 4 (hPDE 4) with which inhibitors interact. One explanation for these observations is that hPDE 4 exists in two distinct forms. One binds the likes of rolipram and denbufylline with a high affinity while the other binds these compounds with a low affinity. The preferred PDE4 inhibitors of use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio is the ratio of the IC₅₀ value for competing with the binding of 1 nM of [³H]R-rolipram to a form of PDE 4 which binds rolipram with a high affinity over the IC₅₀ value for inhibiting the PDE4 catalytic activity of a form which binds rolipram with a low affinity using 1 microM[³H]-cAMP as the substrate. A further review explanation with of this test can be found in co-pending U.S. application Ser. No. 08/456,274 filed 31 May 1995, the text of which is incorporated herein by reference to the extent that text is necessary to the practice of this invention.

Examples of useful PDE4 inhibitors are:
(R)-(+)-1-4-bromobenzyl)-4-[(3-cyclopentyloxy)-4-methoxyphenyl]-2-pyrrolidone;
(R)-(+)-1-(4-bromobenzyl)-4-[(3-cyclopentyloxy)-4-methoxyphenyl]-2-pyrrolidone,
3-(cyclopentyloxy-4-methoxyphenyl)-1-4-N′-[N2-cyano-S-methyl-isothioureido]benzyl)-2-pyrrolidone,
cis 4-cyano-4-(3-cyclopentyloxy-4-methoxyphenyl)cyclohexan-1-carboxylic acid];
cis-[4-cyano-4-(3-cyclopropylmethoxy-4-difluoromethoxyphenyl)cyclohexan-1-ol];
(R)-(+)-ethyl [4-(3-cyclopentyloxy-4-methoxyphenyl)pyrrolidine-2-ylidene)acetate;
(S)(−)-ethyl [4-(3-cyclopentyloxy-4-methoxyphenyl)pyrrolidine-2-ylidene]acetate,

Most preferred are those PDE 4 inhibitors which have an IC₅₀ ratio of greater than 0.5, and particularly those compounds having a ratio of greater than 1.0. Preferred compounds are cis 4-cyano-4-(3-cyclopentyloxymethoxyphenyl)cyclohexan-1-carboxylic acid, 2-carbomethoxy-4-cyano-4-(3-cyclopropylmethoxy-4-difluoromethoxyphenyl)cyclohexan-1-one, and is-[4-cyano-4-(3-cyclopropylmethoxyX difluoromethoxyphenyl)cyclohexan-1-ol]; these are examples of compounds which bind preferentially to the low affinity binding site and which have an IC₅₀ ratio of 0.1 or greater.

Compounds set out in U.S. Pat. No. 5,552,438 issued 03 Sep., 1996. This patent and the compounds it discloses are incorporated herein in full by reference. The compound of particular interest, which is disclosed in U.S. Pat. No. 5,552,438, is cis-4-cyano-4-[3-(cyclopentyloxy)₄-methoxyphenyl]cyclohexane-1-carboxylic acid and its salts, esters, pro-drugs or physical forms.

AWD-12-281 from Astra (Hofgen, N. et al. 15th EFMC Int Symp Med Chem (September 6-10, Edinburgh) 1998, Abst P.98); a 9-benzyladenine derivative nominated NCS-613 (INSERM); D-4418 from Chiroscience and Schering-Plough; a benzodiazepine PDE4 inhibitor identified as CI-1018 (PD-168787; Parke-Davis[Warner-Lambert); a benzodioxole derivative Kyowa Hakko disclosed in WO 9916766; V-11294A from Napp (Landells, L. J. et al. Eur Resp J. [Annu Cong Eur Resp Soc (September 19-23, Geneva) 1998] 1998, 12(Suppl. 28): Abst P2393); roflumilast (CAS reference No 162401-32-3) and a pthalazinone (WO 9947505) from Byk-Gulden; or a compound identified as T-440 (Tanabe Seiyaku; Fuji, K. et al. J. Pharmacol Exp Ther, 1998, 284(1): 162). Any one or all of these compounds may or could benefit from the process described herein.

The several specific compounds set out above which do not have a generic or trade name can be made by the processed described in co-pending U.S. patent applications U.S. Ser. No. 862,083 filed 30 Oct. 1992; U.S. Ser. No. 862, 111 filed 30 Oct. 1992; U.S. Ser. No. 862,030 filed 30 Oct. 1992; and U.S. Ser. No. 862,114 filed 30 Oct. 1992 or their progeny or U.S. patent(s) claiming priority from one or more of these applications. Each of these applications or related patents is incorporated herein by reference in full as if set out in this document.

The steroid agents useful in this invention are oral and inhaled corticosteroids and their pro-drugs which have anti-inflammatory activity. Examples of these steroids are methyl prednisolone, prednisone, dexamethasone, fluticasone, beclomethasone, budesonide, flunisolide, mometasone furoate, and triamcinolone acetonide. Methyl prednisolone and prednisone are oral and injectable forms of anti-inflammatory corticosteroids; they are available from numerous branded and generic pharmaceutical companies. Beclomethasone dipropionate is sold as an aerosol for inhalation under the names Beconase® and Beconase AQ® by Glaxo Wellcome. Fluticasone propionate is sold under the name Flonase® by Glaxo Wellcome as well. Triamcinolone acetonide is sold by Rhone-Poulenc Roher under the name Nasacort® as a nasal spray and aerosol. Flunisolide is sold as a nasal solution under the name Nasalide® and Nasarel™ by Roche Laboratories. Dexamethasone is sold as the sodium phosphate salt by Medeva Pharmaceuticals, Inc. under the name Dexacort™ Phosphate. Mometasone furoate is sold as the monohydrate as a nasal preparation by Schering Corp under the name Nasonex®. Budesonide is yet another inhaled corticosteriod used in treating pulmonary diseases. It is market by Astra Pharmaceuticals, L.P. as a powder in a turbohaler device under the name Pulmicort Turbohaler®. All of these drugs and nasal preparations or oral or injectable formulations can be found in the 1999 edition of the Physicians' Desk Reference® (PDR), published by Medical Economics Corporation, Inc, of New Jersey, USA and is available on the Internet at http://www.tomescps.com/fraainasp?Mnu=0 and linked pages. Additional corticosteroids now under development and which could be used in this invention are set out in Table I.

TABLE I
Inhaled Corticosteroids

| Drug | Company | Indication |
|---|---|---|
| mometasone furoate | Schering-Plough | asthma |
| | | SAR |
| rofleponide | AstraZeneca | asthma |
| ciclesonide | Byk-Gulden/Recordati | asthma |
| butixocort propionate | Warner-Lambert/3M | asthma/rhinitis |
| RPR-106541 | Rhone-Poulenc Rorer | asthma |
| ST-126 | SSP/Torii | asthma |

A preferred combination therapy is that of one or more of dexamethasone, fluticasone, beclomethasone, budesonide, flunisolide, mometasone furoate, and triamcinolone acetonide administered with cis 4-cyano-4-(3-cyclopentyloxy-4-methoxyphenyl)cyclohexan-1-carboxylic acid, cilomalast (Ariflo®). A preferred therapy is concomitant administration of the steroid as an inhalant and the acid in an oral dose form, wherein each drug is administered once or twice a day. In regards to the acid, a controlled-release oral tablet is most preferred.

It is contemplated that both active agents would be administered at the same time, or very close in time. Alternatively, one drug could be taken in the morning and one later in the day. Or in another scenario, one drug could be taken twice daily and the other once daily, either at the same time as one of the twice-a-day dosing occurred, or separately. Preferably both drugs would be taken together at the same time.

The present compounds and pharmaceutically acceptable salts which are active when given orally can be formulated as syrups, tablets, capsules, controlled-release preparation or lozenges. A syrup formulation will generally consist of a suspension or solution of the compound or salt in a liquid carrier for example, ethanol, peanut oil, olive oil, glycerin or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as fluroinated hydrocarbons such as trichlorofluoromethane.

Preferably the composition for the PDE4 inhibitors is a unit dosage form such as a tablet or capsule. For the steroids a metered aerosol dose, metered dry powder inhaler or nasal spray is preferred.

The active ingredient may be administered from 1 to 6 times a day, sufficient to exhibit the desired activity. Preferably, the active ingredient is administered about once or twice a day, more preferably twice a day.

The present compounds are useful for the treatment of exercise-induced asthma (EIA), pollution-induced asthma (PIA) and cold-induced asthma (CIA), both as chronic conditions as well as intermittently, in anticipation of the stimulus in question. Preferably, the present compounds are used for long-term therapy.

As for the amount of drug administered, it is believed that for the PDE4 inhibitors will be administered in an amount of between 1 and 200 micrograms per day per adult human. Steroids can be administered in conformity with approved labeling.

### EXAMPLE 1

### Phosohodiesterase and Rolitram Binding Assays

### EXAMPLE 1A

Isolated human monocyte PDE 4 and hrPDE (human recombinant PDE4) was determined to exist primarily in the low affinity form. Hence, the activity of test compounds against the low affinity form of PDE 4 can be assessed using standard assays for PDE 4 catalytic activity employing 1 microM (3H]cAMP as a substrate (Torphy et al., J. of Biol. Chem., Vol. 267, No. 3 pp 1798-1804, 1992).

Rat brain high speed supernatants were used as a source of protein and both enantionmers of [³H]-rolipram were prepared to a specific activity of 25.6 Ci/mmol. Standard assay conditions were modified from the published procedure to be identical to the PDE assay conditions, except for the last of the cAMP: 50 mM Tris HCl (pH 7.5), 5 mM MgCl₂, 50 microM 5′-AMP and 1 nM of (3H]-rolipram (Torphy et al., J. of Biol. Chem., Vol. 267, No. 3 pp 1798-1804, 1992). The assay was run for 1 hour at 30° C. The reaction was terminated and bound ligand was separated from free ligand using a Brandel cell harvester. Competition for the high affinity binding site was assessed under conditions that were identical to those used for measuring low affinity PDE activity, expect that [³H]-cAMP was not present.

### EXAMPLE 1B

Measurement if Phosphodiesterase Activity

PDE activity was assayed using a [³H]cAMP SPA or [³H]cGMP SPA enzyme assay as described by the supplier (Amersham Life Sciences). The reactions were conducted in 96-well plates it room temperature, in 0.1 ml of reaction buffer containing (final concentrations): 50 mM Tris-HCl. pH 7.5. 8.3 mM MgCl2. 1.7 mM EGTA. [³H]cAMP or [³H]cGMP (approximately 2000 dpm/pmol), enzyme and various concentrations of the inhibitors. The essay was allowed to proceed for 1 hr and was terminated by adding, 50 μl of SPA yttrium silicate beads in the presence of zinc sulfate. The plates were shaken and allowed to stand at room temperature for 20 min. Radiolabeled product formation was assessed be scintillation spectrometry.

[³H]R-rolipram Binding Assay

The [³H]R-rolipram binding assay was performed by modification of the method of Schneider and co-workers, see Nicholson, et al. Trends Pharmacol. Sci. Vol. 12, pp. 19-27 (1991) and McHale et al. Mol. Pharmacol. Vol. 39, 109-113 (1991). R-Rolipram binds to the catalytic site of PDE4 see Torphy et al., Mol. Pharmacol., Vol. 39, pp. 376-384 (1991). Consequently, competition for [³H]R-rolipram binding provides an independent confirmation of the PDE4 inhibitor potencies of unlabeled competitors. The assay was performed at 30° C. for 1 hr in 0.5 ul buffer containing (final concentrations): 50 mM Tris-HCl, pH 7.5, 5 mM MgCl2, 0.05% bovine serum albumin, 2 nM [³H]R-rolipram (5.7×104 dpm/pmol) and various concentrations of non-radiolabeled inhibitors. The reaction was stopped by the addition of 2.5 ml of ice-cold reaction buffer (without [³H]-R-rolipram) and rapid vacuum filtration (Brandel Cell Harvester) through Whatman GF/B filters that had been soaked in 0.3% polyethylenimine. The filters were washed with an additional 7.5-ml of cold buffer, dried, and counted via liquid scintillation spectrometry.

### EXAMPLE 2

Cilomalast/Low Dose Inhaled Corticosteroid (ICS), Dose-Ranging Study

Study Design:

This was a phase IIB, randomised, placebo-controlled, dose-ranging study with a one-week single-blind placebo run-in, a 6-week double-blind treatment phase and a one-week follow-up phase in patients with mild/moderate asthma.

Study population: Male or female patients aged between 18 and 70 years, with mild to moderate asthma, who were not adequately controlled on low doses of inhaled corticosteroids (no greater than 500 mcg beclomethasone dipropionate/day or equivalent) were eligible. Patients were required to have a screening FEV₁ of ≧50% and ≦80% predicted for height, age, sex and race and a 12% or greater reversibility after beta-2 agonist administration. Patients had to have a summary symptom score of 6 or more on 4 out of 7 days preceding the baseline visit to be randomised. The sample size was 300 evaluable patients.

cilomalast was dosed at 5 mg, 10 mg 15 mg twice daily for 6 weeks.

Subjects were on a median of 500 mcg of beclomethasone equivalent although the mean dose of ICS was 652 mcg.

Primary endpoint: change from baseline to endpoint in trough clinic expiratory volume in 1 second (FEV₁), changes in clinic FEV₁ every week and over a 4 hour period following first dose of double-blind medication.

Secondary endpoints: use of rescue medicines and overnight symptoms.

Tertiarty endpoints: clinic forced vital capacity (FVC), clinic peak expiratory flow rate (PEFR), forced expiratory flow at 25-75% (FEF₂₅₋₇₅) and 75% (FEF₇₅), domiciliary PEFR variability, domiciliary PEFR, summary symptom scores.

Evaluation Criteria

The primary efficacy measure was defined as the change from baseline to endpoint in trough clinic forced expiratory volume in 1 second (FEV₁). Changes in clinic FEV₁ were also analysed at each week of the double-blind treatment phase and over a 4-hour period immediately following the first dose of double-blind medication. Secondary efficacy variables were use of inhaled/nebulised beta-2 agonist and overnight asthma symptoms. Tertiary efficacy variables were forced vital capacity (FVC), clinic peak expiratory flow rate (PEFR), forced expiratory flow at 25-75% (FEF₂₅₋₇₅) and 75% (FEF₇₅), domiciliary PEFR variability, domiciliary PEFR (morning and evening), summary symptom score (a composite score of overnight, morning and overall daytime asthma), morning asthma, overall daytime asthma, inhaled corticosteroid use, cough, wheeze, breathlessness/chest tightness, asthma exacerbation rates and global assessments by the physician and the patient.

Study Results:
No statistical improvement in trough FEV₁ at endpoint ITT analysis 15 mg cilomalast BID vs placebo (0.16 L; p=0.062)
Response was dose-ordered for trough FEV₁ at endpoint ITT analysis
Significant improvement in trough FEV₁ (0.21 L; p=0.033) if excluded patients on ICS doses >500 mcg of beclamethasone while on cilomalast 15 mg BID
Corroborating support for cilomalast 15 mg BID included 4 hour FEV1, domicillary PEFR, FEF25-75 and physician and patient global assessment scores

### EXAMPLE 3

### Cilomalast/High Dose Inhaled Corticosteriod (ICS) Study

Study Design:
R, DB, PC, DR in patients with mild/moderate asthma on ≧800 mcg of beclomethasone
cilomalasto doses were 5, 10 and 15 mg twice daily for 4 weeks.
2 week run-in.
Primary endpoint: trough clinic FEV₁.
Secondary endpoints: morning PEFR, symptoms, PEFR variability, evening PEFR, clinic PEFR, FEF25-75, FEF75, rescue medication used.

Study Results:
No statistically significant change in clinic trough FEV₁ in any dose group was noted in the ITT analysis
No dose ordering was observed for the primary endpoint
Trough FEV₁ statistically improved at 10 mg of cilomalast BID using repeated measures analysis is patients excluded on <800 mcg of ICS (0.16 L; p=0.009)
ICS (beclamethasone) dose ranged from 100 to 4000 mcg and averaged 1136 mcg
Corroborative support for the 10 mg of cilomalast BID as the effective dose also came from numerically superior results in the 4 hour clinic FEV₁, clinic FVC, PEFR

No dose-response relationship could be determined, trough concentrations were dose proportional and similar to previous studies.

TABLE 1
Comparison between compounds in selected studies
Allergen challenge studies
Comparative Data: Allergen Challenge Studies

| | | | | ICS | |
|---|---|---|---|---|---|
| | Acitin | | | 800 mcg | |
| | 15 mg/ml | Singulaire | Ultair | budesonide | B2 |
| Max fall LAR | 1.3 | 12.3 | 10.7 | 22.9 | +6.9 |
| treatment | | | | | |
| effect | | | | | |
| Protection | NONE | 36.4 | 30.8 | 32 | NO |
| max fall | | | | | FALL |
| LAR | | | | | |
| [symbol] LAR | 3.5 | 63.5 | 42.4 | not done | |
| treatment | | | | | |
| effect | | | | | |
| Protection | NONE | 56.9 | 42.6 | not done | |
| [symbol] | | | | | |
| LAR | | | | | |

Legend: Data on cilomalast was taken from the protocol analysis in asthma patients on <652 mcg of ICS. Data on montelukast (MT), budesonide (BDP) and both was taken from a single study in the Singulair SBA that examined the effect of Singulair relative to inhaled corticosteroids.

There are significant differences between the behavior of the placebo (Pbo) groups in both studies. Conclusion is that cilomalast outperformed Singulair when added to low dose ICS, despite the fact that the Pbo group also improved in the cilomalast study.

The foregoing statements and examples are intended to illustrate the invention, not to limit it. Reference is made to the claims for what is reserved to the inventors hereunder.

What is claimed is:

1. A method for treating a pulmonary disease by administering to a patient in need thereof an effective amount of a PDE 4 inhibitor and an anti-inflammatory corticosteroid in a combined form, separately, or separately and sequentially where the sequential administration is close in time or remote in time.

2. The method of claim 1 wherein the PDE4 inhibitor is cis 4-cyano-4-(3-cyclopentyloxy-4-methoxyphenyl)cyclohexan-1-carboxylic acid and the steroid is selected from the group consisting of dexamethasone, fluticasone, beclomethasone, budesonide, flunisolide, mometasone furoate, and triamcinolone acetonide.
